(19) United States
(12) Patent Application Publication
(10) Pub. No.: US 2018/0258458 A1
(43) Pub. Date: Sep. 13, 2018

(54) METHOD OF ENRICHING AND DETECTING A TARGET MICROORGANISM

(71) Applicant: 3M INNOVATIVE PROPERTIES COMPANY, St. Paul, MN (US)

(72) Inventor: GREGORY W. SITTON, MINNEAPOLIS, MN (US)

(21) Appl. No.: 15/756,100

(22) PCT Filed: Aug. 29, 2016
(86) PCT No.: PCT/US2016/049182

§ 371 Date: Feb. 28, 2018

Related U.S. Application Data
(60) Provisional application No. 62/213,885, filed on Sep. 3, 2015.

(51) Int. Cl. C12Q 1/04; G01N 33/58; G01N 21/64

(52) U.S. Cl. CPC C12Q 1/04; G01N 33/583; C12Q 2304/24; C12Q 2304/22; G01N 21/6428

(57) ABSTRACT

The present disclosure provides a method of detecting a target bacterium. The method includes forming a primary enrichment culture that includes a test sample, a primary enrichment medium and a redox dye; incubating the primary enrichment culture for a first period of time equal to eight hours or less; assessing the primary enrichment culture during the first period of time to detect whether the redox dye changes from a first state to a second state; if the second state is detected in the primary enrichment culture during the first period of time, mixing a portion of the primary enrichment culture with a secondary enrichment medium to form a secondary enrichment culture and incubating the secondary enrichment culture for a second period of time; and performing a test to detect the target bacterium using a portion of the second enrichment culture.

### CROSS REFERENCE TO RELATED APPLICATIONS

This application claims priority to U.S. Provisional Patent Application No. 62/213,885, filed Sep. 3, 2015, the disclosure of which is incorporated by reference in its entirety herein.

### BACKGROUND

Foodborne illness is a major public health problem. Pathogenic microorganisms, such as species of the Salmonella and Campylobacter genera, account for most foodborne illnesses. The most common sources of Salmonella infections in humans are contaminated foods, including eggs, poultry, produce, meat, and meat products. Eggs and poultry meat are recognized as the major vehicles of human infections because of epizootics in fowl.

Materials (e.g., foods) that contain relatively low numbers of pathogenic microorganisms often also contain nonpathogenic microorganisms. Thus, samples of the material to be tested for pathogenic microorganisms often are subjected to selective enrichment culture techniques in order to increase the relative number of pathogenic microorganisms. Many selective agents are known which, when incorporated into biological growth media, allow for the preferential growth (i.e. selection) of particular organisms, especially particular bacteria.

Various dye substances or salts are used to select for a particular organism (e.g. a pathogen) in a mixed population of bacteria present in a sample obtained from a human or animal subject, as an aid to diagnosis of infectious diseases. However, some selective agents are known to inhibit the growth of healthy cells and to restrict the recovery of injured cells. This is a severe disadvantage because, in many practical applications, it is desired to recover organisms which are injured or “stressed” (e.g. when attempting to recover pathogens from food samples) due to exposure to sub-optimal conditions (of temperature, pH, or the like).

There exists a need for a simple, rapid screening test that identifies presumptively positive Salmonella samples at an early stage of the sample analysis, preferably in a selective enrichment stage of the analysis. Simultaneous enrichment and detection of Salmonella using a single testing method would reduce not only time but also the cost of labor and media. Streamlining procedures and reducing labor and test costs should permit more frequent monitoring for Salmonella, thereby reducing contamination hazard.

### SUMMARY

Certain microbes (e.g., “target” bacteria such as pathogenic bacteria), when present in food or beverages in relatively small quantities, can cause food-borne illness or can multiply to reach concentrations that may cause food-borne illness. Accordingly, it is desirable to have methods that detect very small quantities of microorganisms in a sample. Molecular detection methods afford the capability of detecting relatively small quantities of microorganisms, but they still require threshold concentrations of the microorganisms in order to provide reliable detection. For example, DNA amplification methods typically require greater than or equal to 10³ bacteria per milliliter sample for reliable detection and immunological assays typically require greater than or equal to 10⁵ bacteria per milliliter sample for reliable detection. Thus, in order to detect a concentration of bacteria that is below those threshold levels in a sample (e.g., a food or beverage sample), many methods utilize enrichment culture techniques in order to increase the quantity of target bacteria in the sample.

Enrichment culture techniques often employ selective agents (e.g., salts, antibiotics), the effects to which the target microorganism is normally resistant. However, certain target microorganisms that are otherwise resistant to certain selective agents may be inhibited or killed by the selective agents when the target microorganisms are in a stressed or injured state (e.g., due to heat-, water-, or pH stress). Therefore, it can be advantageous to use a non-selective medium or less-selective medium during a primary enrichment process in order to allow stressed microorganisms to recover. However, the use of less-selective culture medium can permit non-target microorganisms to compete with the target microorganisms for growth-limiting nutrients (e.g., carbon sources, nitrogen sources, energy sources, and/or oxygen). Thus, when the target microorganism is out-numbered by non-target microorganisms in the sample and/or the non-target microorganisms are capable of growing faster than the target microorganisms, it is now known that the sample should be transferred from the less-selective primary enrichment medium to a more-selective secondary enrichment medium before the non-target microorganisms overwhelmingly out-number the target microorganism. The present disclosure provides a simple method to mitigate a possible negative effect of having large numbers of non-target microorganisms in a sample on the detection of a relatively low number of target microorganisms in the sample.

In one aspect, the present disclosure provides a first method of detecting a bacterium belonging to the genus Salmonella. The method can comprise mixing a first volume of a test sample with a second volume of a primary enrichment medium to form a primary enrichment culture, wherein the primary enrichment culture comprises a redox dye; incubating the primary enrichment culture for a first period of time equal to eight hours or less; assessing the primary enrichment culture during the first period of time to detect whether the redox dye changes from a first state to a second state; if the second state is detected in the primary enrichment culture during the first period of time, mixing a first portion of the primary enrichment culture with a secondary enrichment medium to form a secondary enrichment culture and incubating the secondary enrichment culture for a second period of time; and performing a test to detect the bacterium of the genus Salmonella using a second portion of the second enrichment culture.

In another aspect, the present disclosure provides a second method of detecting a bacterium belonging to the genus Salmonella. The method can comprise mixing a first volume of a test sample with a second volume of a primary enrichment medium to form a primary enrichment culture, wherein the primary enrichment culture comprises a redox dye; incubating the primary enrichment culture for a first period of time equal to eight hours or less; assessing the primary enrichment culture during the first period of time to detect whether the redox dye changes from a first state to a second state; if the second state is not detected in the primary enrichment culture during the first period of time, incubating the primary enrichment culture for a third period of time; and performing a test to detect the bacterium of the genus Salmonella using a third portion of the first enrichment culture.

In any of the above embodiments of the first method or second method, performing a test to detect the bacterium from the genus Salmonella can comprise performing a test selected from the group consisting of a test for detecting an immunological test to detect an antigen associated with the bacterium from the genus Salmonella, a genetic test to detect a nucleic acid sequence associated with the bacterium from the genus Salmonella, and a combination of any two or more of the foregoing tests.

In yet another aspect, the present disclosure provides a third method of detecting a target bacterium. The method can comprise mixing a first volume of a test sample with a second volume of a primary enrichment medium to form a primary enrichment culture, wherein the primary enrichment culture comprises a redox dye; incubating the primary enrichment culture for a first period of time equal to eight hours or less; assessing the primary enrichment culture during the first period of time to detect whether the redox dye changes from a first state to a second state; if the second state is detected in the primary enrichment culture during the first period of time, mixing a first portion of the primary enrichment culture with a secondary enrichment medium to form a secondary enrichment culture and incubating the secondary enrichment culture for a second period of time; and performing a test to detect the target bacterium using a second portion of the second enrichment culture.

In any of the above embodiments of the methods, the redox dye can be selected from the group consisting of methylene blue, resazurin, and a tetrazolium dye. In any of the above embodiments of the methods, the primary enrichment medium can be preheated. In any of the above embodiments of the methods, the first state can be a first color and the second state can be a second color that is measurably different than first color.

In any of the above embodiments of the methods, incubating the primary enrichment culture for a first period of time equal to eight hours or less can comprise incubating the primary enrichment culture for a first period of time equal to six hours or less. In any of the above embodiments of the methods, assessing the primary enrichment culture during the first period of time can comprise assessing the primary enrichment culture at a first time point and assessing the primary enrichment culture at a second time point after the first time point.

In any of the above embodiments of the third method or the fourth method, performing a test to detect the target bacterium can comprise performing a test selected from the group consisting of a test for detecting an immunological test to detect an antigen associated with the target bacterium, a genetic test to detect a nucleic acid sequence associated with the target bacterium, and a combination of any two or more of the foregoing tests.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

As used herein, “a,” “an,” “the,” “at least one,” and “one or more” are used interchangeably. Thus, for example, a nutrient can be interpreted to mean “one or more” nutrients.

The term “and/or” means one or all of the listed elements or a combination of any two or more of the listed elements.

Also herein, the recitations of numerical ranges by endpoints include all numbers subsumed within that range (e.g., 1 to 5 includes 1, 1.5, 2, 2.75, 3, 3.80, 4, 5, etc.).

The term “microorganism” or “microbe” as used herein refers to any microscopic organism, which may be a single cell or multicellular organism. The term is generally used to refer to any prokaryotic or eukaryotic microscopic organism capable of growing and reproducing in a suitable culture medium, including without limitation, one or more of bacteria. Microorganisms encompassed by the scope of the present invention include prokaryotes, namely the bacteria and archaea; and various forms of eukaryotes, comprising the protozoa, fungi, yeast (e.g., anaerobic yeast), algae etc. The term “target microorganism” refers any microorganism that is desired to be detected.

The term “culture” or “growth” of microorganisms as used herein refers to the method of multiplying microbial organisms by letting them reproduce in predetermined culture media under conditions conducive for their growth. More particularly it is the method of providing a suitable culture medium and conditions to facilitate at least one cell division of a microorganism. Culture media are solid, semisolid or liquid media containing all of the nutrients and necessary physical growth parameters necessary for microbial growth.

The term “enrichment” as used herein refers to the culture method of selectively enriching the growth of a specific microorganism by providing medium and conditions with specific and known attributes that favors the growth of that particular microorganism. The enrichment culture's environment will positively influence the growth of a selected microorganism and/or negatively influence the growth of other microorganisms.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

Additional details of these and other embodiments are set forth in the accompanying drawings and the description below. Other features, objects and advantages will become apparent from the description and drawings, and from the claims.

### BRIEF DESCRIPTION OF DRAWINGS

FIG. 1 is a block diagram showing one embodiment of a method according to the present disclosure.

FIG. 2 is a graph showing the relationship between the initial concentration of colony-forming units (CFU) in a primary enrichment culture and the amount of time it takes to convert a redox dye from a first state to a second state.

### DETAILED DESCRIPTION

Before any embodiments of the present disclosure are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “connected” and “coupled” and variations thereof are used broadly and encompass both direct and indirect connections and couplings. Further, “connected” and “coupled” are not restricted to physical or mechanical connections or couplings. It is to be understood that other embodiments may be utilized and structural or logical changes may be made without departing from the scope of the present disclosure. Furthermore, terms such as “front,” “rear,” “top,” “bottom,” and the like are only used to describe elements as they relate to one another, but are in no way meant to recite specific orientations of the apparatus, to indicate or imply necessary or required orientations of the apparatus, or to specify how the invention described herein will be used, mounted, displayed, or positioned in use.

The present disclosure generally relates to the detection of target microorganisms in a sample. In particular, the present disclosure relates to a method that involves enrichment culture of target microorganisms so that relatively low numbers (e.g., ≤100 microorganisms per milliliter, ≤10 microorganisms per milliliter, ≤1 microorganism per milliliter, ≤0.1 microorganism per milliliter, ≤0.01 microorganism per milliliter, <=0.004 microorganism per milliliter (i.e., a detection limit of 1 microorganism/250 mL), <0.0003 microorganisms per milliliter (i.e., a detection limit of 1 microorganism/3500 mL)) can be detected in a sample that also contains relatively higher quantities of non-target microorganisms (e.g., ≥10 non-target microorganisms per target microorganism, ≥100 non-target microorganisms per target microorganism, ≥1000 non-target microorganisms per target microorganism, ≥10000 non-target microorganisms per target microorganism, ≥10⁷ non-target microorganisms per target microorganism).

Methods of the present disclosure employ the use of early detection of relatively large concentrations of growing microorganisms (i.e., target and non-target growing microorganisms) in a primary enrichment medium in order to determine whether more-selective secondary enrichment media should be used in order to facilitate growth and detection of the target microorganisms.

In one aspect, the present disclosure provides a method. The method can be used to detect a target microorganism such as a bacterium, a yeast, or a mold in a sample that contains at least one non-target microorganism that may compete against the target microorganism for a growth-limiting nutrient in a liquid culture medium that is used to cultivate, in order to detect, relatively low quantities of the target microorganism in the sample. The target microorganism can be a microorganism of particular interest such as, for example, a pathogenic bacterium that may be found in the sample and that may indicate a risk of infection or intoxication associated with the material or environment from which the sample was obtained. Materials that may be tested according to the methods of the present disclosure include, for example, raw materials for making food or beverage products, in-process materials (including process water and the like) in food or beverage production, finished goods from food or beverage production, or environmental samples taken from a food or beverage production or storage facility.

Microorganisms of particular interest include prokaryotic and eukaryotic organisms, particularly Gram positive bacteria, Gram negative bacteria, filamentous fungi, and yeast. Exemplary target microorganisms include, but are not limited to, members of the family Enterobacteriaceae, or the family Micrococcaceae or the genera Staphylococcus spp., Streptococcus spp., Pseudomonas spp., Enterococcus spp., Salmonella spp., Legionella spp., Shigella spp. Yersinia spp., Enterobacter spp., Escherichia spp., Bacillus spp., Listeria spp., Vibrio spp., Corynebacteria spp., as well as Aspergillus spp., Fusarium spp., Cronobacter spp, and Candida spp. Particularly virulent organisms include Staphylococcus aureus, S. epidermidis, Streptococcus pneumoniae, S. agalactiae, S. pyogenes, Enterococcus faecalis, Bacillus anthracis, Pseudomonas aeruginosa, Escherichia coli, Aspergillus niger, A. fumigatus, A. clavatus, Fusarium solani, F. oxysporum, F. chlamydosporum, Listeria monocytogenes, Listeria ivanovii, Vibrio cholera, V. parahemolyticus, Salmonella cholerasuis, S. typhi, S. typhimurium, Candida albicans, C. glabrata, C. krusei, Cronobacter sakazakii, and E. coli O157.

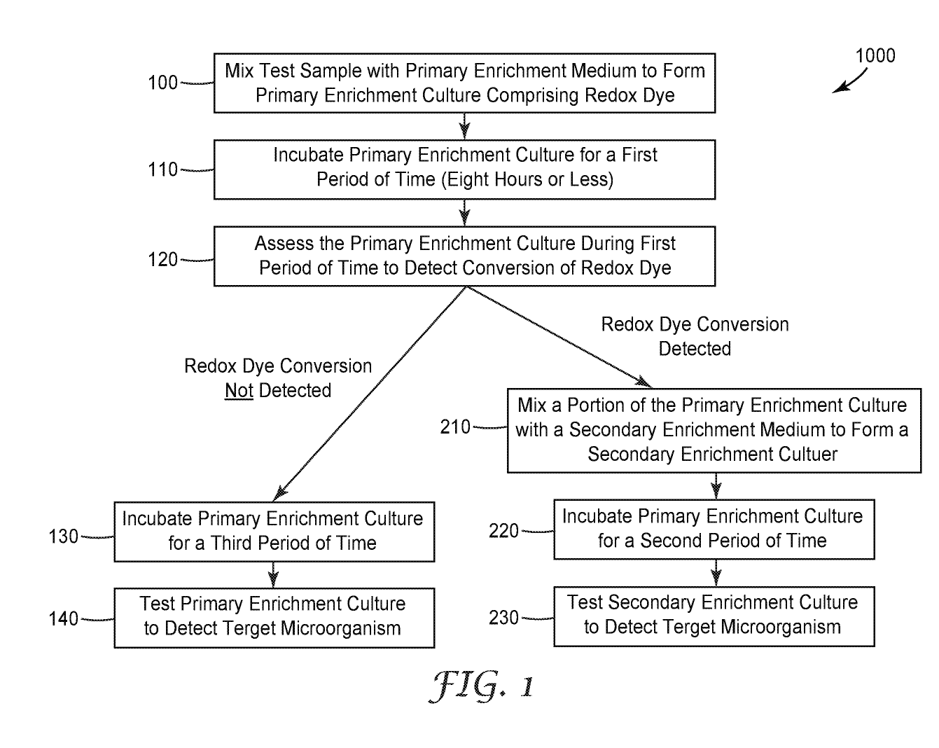

In any embodiment, the method can be used to detect a target microorganism (e.g., a target bacterium such as Salmonella bacterium, for example) in a sample. FIG. 1 shows a block diagram of one embodiment of a method 1000 of detecting a target bacterium according to the present disclosure. The method 1000 comprises a step 100 of mixing a first volume of a test sample with a second volume of a primary enrichment medium to form a primary enrichment culture. The primary enrichment culture comprises a redox dye. The redox dye can be any redox dye that is suitable for providing a detectable (e.g., visually-detectable) indication of microorganism metabolic activity in a liquid culture medium. Redox dyes that provide a visually-detectable indication of microbial activity are known in the art and include; but are not limited to; methylene blue, resazurin, and tetrazolium dyes. A non-limiting example of a suitable tetrazolium dye is triphenyl tetrazolium chloride (“TTC”).

In any embodiment, the primary enrichment medium is selected to provide a nutrient environment that promotes growth of the target microorganism. The primary enrichment medium may be somewhat selective (e.g., by its composition and/or pH) such that it favors growth of the target microorganism over at least one other microorganism that may be present in the test sample. Preferably, the primary enrichment medium facilitates growth of all target microorganism, including stressed or injured target microorganisms. Selection of the primary enrichment medium is made according to the target microorganism to be detected. A nonlimiting example of a suitable primary enrichment medium for detecting a Salmonella microorganism is buffered peptone water. Other nonlimiting examples of suitable primary enrichment medium for growth and detection of bacteria (including Salmonella bacteria) include Brain Heart Infusion broth, non-fat dry milk, lactose broth, Buffered Peptone Water (ISO), modified Tryptone Soya Broth, and Universal Pre-enrichment Broth.

The primary enrichment medium can be provided as a dry powder, an agglomerated powder, or as a liquid medium. Optionally, a liquid primary enrichment medium can be a concentrated medium that is diluted to a working concentration by the first volume of the test sample. In any embodiment, the test sample may comprise a component of the primary enrichment medium (e.g., a nutrient, a selective agent such as a salt or an antibiotic, for example).

In any embodiment, prior to mixing the test sample with the primary enrichment medium, the primary enrichment medium may optionally be preheated. For example, when the target microorganism is a Salmonella microorganism, the primary enrichment medium may be preheated to a temperature of about 30° C., about 31° C., about 32° C., about 33° C., about 34° C., about 35° C., about 36° C., about 37° C., about 38° C., about 39° C., about 40° C., about 41° C., about 42° C., or about 43° C. In any embodiment, the primary enrichment medium may be preheated to a temperature between 35° C. and 43° C., inclusive. In any embodiment, the primary enrichment medium may be preheated to a temperature above 43° C., provided the mixing step does not expose a target microorganism in the sample to a temperature environment that is lethal to the target microorganism (e.g., a Salmonella microorganism). Advantageously, preheating the primary enrichment medium reduces the time it takes to adjust the temperature of the test sample to a temperature at which the primary enrichment culture is incubated.

In any embodiment, mixing a first volume of a test sample with a second volume of a primary enrichment medium comprises contacting first and second volumes that are substantially equal. Alternatively, mixing a first volume of a test sample with a second volume of a primary enrichment medium comprises mixing first and second volumes that are not equal. For example, the first volume may be smaller than the second volume or the first volume may be larger than the second volume. In any embodiment, a ratio of the first volume to the second volume can be less than or equal to 10:1, less than or equal to 5:1, less than or equal to 3:1, less than or equal to 2:1, or less than or equal to 1:1. When the ratio of the first volume to the second volume is greater than 1:1, optionally, the primary enrichment medium can be provided as a concentrate that is diluted to an appropriate working concentration when mixed with a predefined first volume of the test sample.

Referring back to FIG. 1, the method 1000 further comprises a step 110 of incubating the primary enrichment culture for a first period of time equal to eight hours or less. Incubating the primary enrichment culture comprises holding the primary enrichment culture at a temperature that facilitates growth of the target microorganism. The preferred incubation temperatures for a particular target microorganism in a particular primary enrichment medium are well known to a person having ordinary skill in the art. For example, in order to detect a Salmonella microorganism, the primary enrichment medium may be buffered peptone water and the incubation temperature used for the first period of time may be about 35° C. to about 43° C., inclusive (e.g., about 30° C., about 31° C., about 32° C., about 33° C., about 34° C., about 35° C., about 36° C., about 37° C., about 38° C., about 39° C., about 40° C., about 41° C., about 42° C., about 42.5° C., or about 43° C.).

The first period of time is a quantity of time suitable to determine whether the test sample has such a large number of non-target microorganisms that they (non-target microorganisms) may interfere with growth of the target microorganisms in the primary enrichment culture. It is now know that, if that condition is present (i.e., there is a large number of non-target microorganisms in the test sample), at least a portion of the primary enrichment culture should be mixed with a more-selective secondary enrichment medium in order to suppress growth of at least some of the non-target microorganisms, thereby permitting growth and detection of the target microorganisms as described herein.

The first period of time may be selected according to several factors known to a person having ordinary skill in the art. These factors include, for example, the composition (e.g., nutrients, selective agents, pH) of the primary enrichment medium, the incubation temperature, the typical doubling time of microorganisms (including non-target microorganisms) in the particular primary nutrient medium at the particular incubation temperature, and the likelihood of having stressed microorganisms in the test sample. In any embodiment, the first period of time can be about 12 hours or less; preferably, about 10 hours or less; and more preferably, about 8 hours or less. In any embodiment, including an embodiment of the method for detection of a Salmonella microorganism, the first period of time is less than or equal to 8 hours, less than or equal to 7 hours, less than or equal to 6 hours, less than or equal to 5 hours, less than or equal to 4 hours, less than or equal to 3 hours, less than or equal to 2 hours, less than or equal to 1 hour, or less than or equal to 0.5 hours.

In any embodiment, incubating the primary enrichment culture for a first period optionally comprises incubating the primary enrichment culture with agitation. Incubating with agitation may facilitate aeration of the culture medium, which may additionally facilitate growth of the target microorganism. In addition, aeration of the culture medium further may provide the target microorganism a selective growth advantage over non-target microorganisms present in the test sample.

Referring back to FIG. 1, the method 1000 further comprises the step 120 assessing the primary enrichment culture during the first period of time to detect whether the redox dye changes from a first state to a second state. The primary enrichment culture can be assessed, for example, by visually observing the medium to detect a color associated with the redox indicator.

In any embodiment of the methods of the present disclosure, the redox dye has a first state (e.g., oxidized state) and a second state (e.g., reduced state). In its first state, the redox dye or derivative thereof has a first color and; in its second state, the redox dye or derivate thereof second state has a second color that is measurably (e.g., via optical techniques) different than first color. The measurable difference between the first color and the second color can relate to the intensity and/or the wavelength. In addition, with certain redox dyes, the first state can be substantially more-colored (e.g., have a greater optical density) than the second state or the first state can be substantially less-colored (e.g., have a lesser optical density) than the second state. In some embodiments, “substantially less-colored” means the redox dye is visually colorless.

For example, the redox indicator (e.g., methylene blue) has a visually-detectable blue color when it is present in an aqueous liquid in a first state (e.g., an oxidized state) and is colorless when it is present in the aqueous liquid in a second state (e.g., a reduced state). When oxidized methylene blue is present in a microbial culture medium, the natural color of the medium is tinted (e.g., blue or blue-green) by the blue color of the oxidized methylene blue. A variety of microorganisms are capable of reducing methylene blue from the first state to the second state (which is visually colorless) during normal metabolism of the nutrients in the culture medium, thereby eliminating the blue tint in the culture medium (i.e., the culture medium reverts to a color that is closer to its natural color as if contains a low concentration of the oxidized form of methylene blue).

Alternatively, the redox indicator (e.g., TTC) is substantially colorless in its first state (oxidized state) and causes an enrichment containing TTC to change from its natural color (e.g., straw-colored) to a color that includes a visually-detectable red tint when the TTC is reduced by microbial activity and the reduced form of TTC spontaneously polymerizes to produce formazan.

Although using a redox indicator that forms a visually-detectable change in the presence of large numbers of bacteria is a simple way of detecting the change of the redox dye, in any embodiment, detecting a change in the redox indicator can alternatively be performed using an instrument (e.g., a spectrophotometer, a fluorometer) to measure light absorbance, reflectance, or emission by the liquid medium of the primary enrichment culture.

Assessing the primary enrichment culture during the first period of time to detect whether the redox dye changes from a first state to a second state according to the present disclosure typically comprises comparing the instantaneous state of the redox dye in the primary enrichment culture to a reference in order to determine whether (or to what extent) the redox dye has changed from the first state to the second state. The reference may comprise, for example, an otherwise identical composition that contains no test sample (i.e., the reference contains the primary enrichment medium, the redox dye, and a volume of suitable sterile diluent equivalent to the first volume of test sample in the primary enrichment culture. Alternatively, the reference may be an optical image or reading of the enrichment culture taken at “T=0” (i.e., at the moment a mixture comprising the enrichment medium, the test sample, and the redox dye is formed) or the reference may be a printed card that has a printed color selected to match the color of the mixture comprising the test sample, primary enrichment medium, and the redox dye.

The primary enrichment culture can be assessed at any time during the first period of time, including the “T=0” time (e.g., immediately after the test sample, primary enrichment medium, and the redox dye are mixed) or at the end of the first time period (e.g., 6 hours after the test sample, primary enrichment medium, and the redox dye are mixed).

In any embodiment of a method according to the present disclosure, assessing the primary enrichment culture during the first period of time comprises assessing the primary enrichment culture at a first time point and assessing the primary enrichment culture at a second time point after the first time point. The first time point can be the T=0 time, for example, and the second time point can be any other time point during the first time period up to and including the end of the first time period. Advantageously, assessing the primary enrichment culture at a plurality of time points during the first time period permits the operator to observe a detectable change of the redox dye at a time that may be less than the end point of the first time period. If the detectable change occurs at or before the end point of the first time period, the operator can act on the observation according to the method of the present disclosure.

Referring back to FIG. 1, the next step in the method 1000 is dependent upon whether a conversion of the redox indicator from the first state to the second state is detected during the first time period. If a conversion of the redox dye is detected during the first time period, the method comprises the step 210 of mixing a first portion of the primary enrichment culture with a secondary enrichment medium to form a secondary enrichment culture and incubating the secondary enrichment culture for a second period of time. The first portion of the primary enrichment culture can be any portion (up to 100%) of the entire volume of the primary enrichment culture. A person having ordinary skill in the art will recognize that the quantity of test sample present in the first portion will directly affect the overall sensitivity (i.e., limit of detection) of the method. For example, if the primary enrichment culture contains 10 mL of test sample and the first portion consists of 10% of the primary enrichment culture, the overall sensitivity of the method is about 1 target microorganism per milliliter of test sample.

Mixing a first portion of the primary enrichment culture with a secondary enrichment medium typically comprises mixing the first portion with a secondary enrichment medium that is more-selective for the target microorganism than the primary enrichment medium. That is, the secondary enrichment medium includes nutrients and/or selective agents (e.g., salts, fatty acids, hydrogen ion concentration, antibiotics, or combinations thereof) that inhibit growth of non-target microorganisms, relative to target microorganisms, more stringently than the primary enrichment medium. The secondary enrichment medium can be provided as a dry powder or agglomerated powder or as a liquid medium. Optionally, a liquid secondary enrichment medium can be a concentrated medium that is diluted to a working concentration by the first portion of the primary enrichment culture. In any embodiment, the secondary enrichment medium optionally may be more-selective for the target microorganism than the primary enrichment medium (i.e., the secondary enrichment medium favors—to a degree that is greater than the primary enrichment medium—the growth of the target microorganism over at least one non-target microorganism).

After forming the secondary enrichment culture, the method further comprises the step 220 of incubating the secondary enrichment culture for a second period of time. Incubating the secondary enrichment culture comprises holding the secondary enrichment culture at a temperature that facilitates growth of the target microorganism. The preferred incubation temperatures for a particular target microorganism in a particular secondary enrichment medium are well known to a person having ordinary skill in the art. For example, in order to detect a Salmonella microorganism, the secondary enrichment medium may be Rappaport-Vassiliadis broth, Rappaport-Vassiliadis Soya Peptone broth, Rappaport-Vassiliadis R10 broth, Selenite Cystine broth, Tetrathionate broth (“TT broth”), Muller-Kaufmann Tetrathionate broth, or TT broth (Hanja) and the incubation temperature used for the second period of time may be about 35° C. to about 43° C., inclusive (e.g., about 30° C., about 31° C., about 32° C., about 33° C., about 34° C., about 35° C., about 36° C., about 37° C., about 38° C., about 39° C., about 40° C., about 41° C., about 42° C., about 42.5° C., or about 43° C.).

The second period of time is a quantity of time suitable to permit at least one cell division and, preferably, a plurality of cell divisions by any target microorganism present in the primary enrichment culture.

The second period of time may be selected according to several factors known to a person having ordinary skill in the art. These factors include, for example, the composition (e.g., nutrients, selective agents, pH) of the secondary enrichment medium, the incubation temperature, and the typical doubling time of microorganisms (especially, the target microorganisms) in the particular secondary nutrient medium at the particular incubation temperature. In any embodiment, the second period of time can be about 26 hours or less. In any embodiment, the second period of time can be about 10-48 hours, about 12-26 hours, or about 18-26 hours. In any embodiment, including an embodiment of the method for detection of a Salmonella microorganism, the second period of time is about 10-48 hours, about 10-24 hours, or about 10-18 hours.

In any embodiment, incubating the secondary enrichment culture for a second period optionally comprises incubating the secondary enrichment culture with agitation. Incubating with agitation may facilitate aeration of the culture medium, which may additionally facilitate growth of the target microorganism. In addition, aeration of the culture medium further may provide the target microorganism a selective growth advantage over non-target microorganisms present in the test sample.

After incubating the secondary enrichment culture for the second period of time, the method 1000 includes the step 230 of performing a test to detect the target microorganism using a second portion of the secondary enrichment culture. Although the second portion can be any portion (up to 100%) of the entire volume of the secondary enrichment culture, it is preferable to test a portion that is less than 100%. In any embodiment, the second portion may be substantially less than 100% (e.g., less than or equal to 10%, less than or equal to 5%, less than or equal to 1%, less than or equal to 0.1%, less than or equal to 0.01%). A person having ordinary skill in the art will recognize that the quantity of test sample present in the second portion will directly affect the overall sensitivity (i.e., limit of detection) of the method. For example, if the primary enrichment culture contains 10 mL of test sample, the first portion consists of 10% of the primary enrichment culture, and the second portion consists of 0.1% of the secondary enrichment culture, the overall sensitivity of the method is about 1000 target microorganisms per milliliter of test sample.

The second portion can be tested to detect the target microorganism using any detection method known in the art that is compatible for use with enrichment cultures. Suitable detection tests may include additional sample preparation steps (e.g., a wash step, a cell concentration step, a cell lysis step, and the like) in order to prepare the sample to detect the target microorganism. Suitable tests to detect the target microorganism include, but are not limited to, a test for detecting a predetermined enzyme activity associated with the target microorganism, an immunological test to detect an antigen associated with the target microorganism, a genetic test to detect a nucleic acid sequence associated with the target microorganism, culturing the microorganism on or in a semisolid medium, and a combination of any two or more of the foregoing tests. A preferred detection test used to detect a target microorganism in a method according to the present disclosure includes the isothermal nucleic acid detection technology sold by 3M Company (St. Paul, Minn.) under the trade name 3M™ Molecular Detection System.

If a conversion of the redox dye is not detected during the first time period, the method 1000 comprises the step 130 of incubating the primary enrichment culture for a third period of time. Incubating the primary enrichment culture for a third period of time comprises holding the primary enrichment culture at a temperature that facilitates growth of the target microorganism. The incubation temperature for the third period of time can be the same incubation temperature used during the first period of time or it may be different from the temperature used during the first period of time. The preferred incubation temperatures for a particular target microorganism in a particular primary enrichment medium are well known to a person having ordinary skill in the art. For example, in order to detect a Salmonella microorganism, the incubation temperature used for the third period of time may be about 35° C. to about 43° C., inclusive (e.g., about 30° C., about 31° C., about 32° C., about 33° C., about 34° C., about 35° C., about 36° C., about 37° C., about 38° C., about 39° C., about 40° C., about 41° C., about 42° C., about 42.5° C., or about 43° C.).

The third period of time is a quantity of time suitable to permit at least one cell division and, preferably, a plurality of cell divisions by any target microorganism present in the primary enrichment culture.

The third period of time may be selected according to several factors known to a person having ordinary skill in the art. These factors include, for example, the composition (e.g., nutrients, selective agents, pH) of the primary enrichment medium, the incubation temperature, and the typical doubling time of microorganisms (especially, the target microorganisms) in the particular primary enrichment medium at the particular incubation temperature. In any embodiment, the third period of time can be about 26 hours or less. In any embodiment, the third period of time can be about 10-48 hours, about 12-26 hours, or about 18-26 hours. In any embodiment, including an embodiment of the method for detection of a Salmonella microorganism, the third period of time is about 10-48 hours, about 10-24 hours, or about 10-18 hours.

In any embodiment, incubating the primary enrichment culture for a third period optionally comprises incubating the primary enrichment culture with agitation. Incubating with agitation may facilitate aeration of the culture medium, which may additionally facilitate growth of the target microorganism. In addition, aeration of the culture medium further may provide the target microorganism a selective growth advantage over non-target microorganisms present in the test sample.

After incubating the secondary enrichment culture for the second period of time, the method 1000 comprises the step 140 of performing a test to detect the target microorganism using a third portion of the first enrichment culture. Although the third portion can be any portion (up to 100%) of the entire volume of the primary enrichment culture, it is preferable to test a portion that is less than 100%. In any embodiment, the third portion may be substantially less than 100% (e.g., less than or equal to 10%, less than or equal to 5%, less than or equal to 1%, less than or equal to 0.1%, less than or equal to 0.01%). A person having ordinary skill in the art will recognize that the quantity of test sample present in the third portion will directly affect the overall sensitivity (i.e., limit of detection) of the method. For example, if the primary enrichment culture contains 10 mL of test sample and the third portion consists of 0.1% of the primary enrichment culture, the overall sensitivity of the method is about 100 target microorganisms per milliliter of test sample.

The third portion can be tested to detect the target microorganism using any detection method known in the art that is compatible for use with enrichment cultures. Suitable detection tests may include additional sample preparation steps (e.g., a wash step, a cell concentration step, a cell lysis step, and the like) in order to prepare the sample to detect the target microorganism. Suitable tests to detect the target microorganism include, but are not limited to, a test for detecting a predetermined enzyme activity associated with the target microorganism, an immunological test to detect an antigen associated with the target microorganism, a genetic test to detect a nucleic acid sequence associated with the target microorganism, culturing the bacterium on or in a semisolid medium, and a combination of any two or more of the foregoing tests. A preferred detection test used to detect a target microorganism in a method according to the present disclosure includes the isothermal nucleic acid detection technology sold by 3M Company (St. Paul, Minn.) under the trade name 3M™ Molecular Detection System.

In yet another aspect, the present disclosure provides a method of enriching a target microorganism in a liquid culture medium. The method is particularly preferred when an operator suspects the target microorganism is present in a material such as, for example, the materials described herein. Thus, the method can be used to enrich the target microorganism from a test sample of the material.

The method comprises mixing a first volume of a test sample with a second volume of a primary enrichment medium to form a primary enrichment culture as described herein. The primary enrichment medium comprises a redox dye as described herein. The method further comprises incubating the primary enrichment culture for a first period of time equal to eight hours or less and assessing the primary enrichment culture during the first period of time to detect whether the redox dye changes from a first state to a second state, each as described herein.

### Exemplary Embodiments

Embodiment A is a method of detecting a bacterium belonging to the genus Salmonella, the method comprising:

mixing a first volume of a test sample with a second volume of a primary enrichment medium to form a primary enrichment culture;
wherein the primary enrichment culture comprises a redox dye;

incubating the primary enrichment culture for a first period of time equal to eight hours or less;

assessing the primary enrichment culture during the first period of time to detect whether the redox dye changes from a first state to a second state;

if the second state is detected in the primary enrichment culture during the first period of time, mixing a first portion of the primary enrichment culture with a secondary enrichment medium to form a secondary enrichment culture and incubating the secondary enrichment culture for a second period of time; and

performing a test to detect the bacterium of the genus Salmonella using a second portion of the second enrichment culture.

Embodiment B is a method of detecting a bacterium belonging to the genus Salmonella, the method comprising:

mixing a first volume of a test sample with a second volume of a primary enrichment medium to form a primary enrichment culture;
wherein the primary enrichment culture comprises a redox dye;

incubating the primary enrichment culture for a first period of time equal to eight hours or less;

assessing the primary enrichment culture during the first period of time to detect whether the redox dye changes from a first state to a second state;

if the second state is not detected in the primary enrichment culture during the first period of time, the method further comprises incubating the primary enrichment culture for a third period of time; and

performing a test to detect the bacterium of the genus Salmonella using a third portion of the primary enrichment culture.

Embodiment C is the method of Embodiment A or Embodiment B, wherein the redox dye is selected from the group consisting of methylene blue, resazurin, and a tetrazolium dye.

Embodiment D is the method of any one of the preceding Embodiments, wherein a ratio of the first volume to the second volume is less than or equal to 10:1.

Embodiment E is the method of any one of the preceding Embodiments, wherein the primary enrichment medium is preheated.

Embodiment F is the method of Embodiment E, wherein the primary enrichment medium is preheated to 35-45°, inclusive.

Embodiment G is the method of any one of the preceding Embodiments, wherein incubating the primary enrichment culture for a first period of time comprises incubating the primary enrichment culture at 37-42° C., inclusive.

Embodiment H is the method of any one of the preceding Embodiments, wherein incubating the secondary enrichment culture for a second period of time comprises incubating the secondary enrichment culture at 37-42° C., inclusive.

Embodiment I is the method of any one of the preceding Embodiments, wherein incubating the primary enrichment culture for a third period of time comprises incubating the primary enrichment culture at 37-42° C., inclusive.

Embodiment J is the method of any one of the preceding claims, wherein the first state has a first color and the second state has a second color that is measurably different than first color.

Embodiment K is the method of Embodiment J, wherein the first state is a colorless state and the second state is a colored state.

Embodiment L is the method of Embodiment J, wherein the first state is a colored state and the second state is a colorless state.

Embodiment M is the method of any one of the preceding Embodiments, wherein the test sample comprises a component of the primary enrichment medium.

Embodiment N is the method of any one of the preceding Embodiments, wherein the first period of time is equal to or less than 4 hours.

Embodiment O is the method of Embodiment N, wherein the first period of time is equal to or less than 2 hours.

Embodiment P is the method of any one of the preceding Embodiments, wherein assessing the primary enrichment culture during the first period of time comprises visually assessing the primary enrichment culture.

Embodiment Q is the method of any one of the preceding Embodiments, wherein assessing the primary enrichment culture during the first period of time comprises assessing the primary enrichment culture at a first time point and assessing the primary enrichment culture at a second time point after the first time point.

Embodiment R is the method of Embodiment Q, wherein assessing the primary enrichment culture during the first period of time comprises assessing the primary enrichment culture for the first state of the redox dye at the first time point and assessing the primary enrichment culture for the second state of the redox dye at the second time point.

Embodiment S is the method of any one of the preceding Embodiments, wherein assessing the primary enrichment culture during the first period of time comprises using an instrument to measure light absorbance or reflectance by the primary enrichment culture.

Embodiment T is the method of any one of the preceding Embodiments, wherein the second period of time is about 10 hours to about 48 hours, inclusive.

Embodiment U is the method of Embodiment T, wherein the second period of time is about 10 hours to about 26 hours, inclusive.

Embodiment V is the method of Embodiment U, wherein the second period of time is about 18 hours to about 26 hours, inclusive.

Embodiment W is the method of any one of Embodiments B though S, wherein the third period of time is about 10 hours to about 48 hours, inclusive.

Embodiment X is the method of Embodiment W, wherein the third period of time is about 10 hours to about 18 hours, inclusive.

Embodiment Y is the method of any one of the preceding Embodiments, wherein performing a test to detect the bacterium from the genus Salmonella comprises performing a test selected from the group consisting of a test for detecting a predetermined enzyme activity associated with the bacterium from the genus Salmonella, an immunological test to detect an antigen associated with the bacterium from the genus Salmonella, a genetic test to detect a nucleic acid sequence associated with the bacterium from the genus Salmonella, culturing the bacterium on or in a semisolid medium, and a combination of any two or more of the foregoing tests.

Embodiment Z is the method of any one of the preceding Embodiments, wherein the primary enrichment medium is a liquid medium.

Embodiment AA is the method of Embodiment Z, wherein the secondary enrichment medium is a liquid medium.

Embodiment AB is a method of detecting a target bacterium, the method comprising:

mixing a first volume of a test sample with a second volume of a primary enrichment medium to form a primary enrichment culture;
wherein the primary enrichment culture comprises a redox dye;

incubating the primary enrichment culture for a first period of time equal to eight hours or less;

assessing the primary enrichment culture during the first period of time to detect whether the redox dye changes from a first state to a second state;

if the second state is detected in the primary enrichment culture during the first period of time, mixing a first portion of the primary enrichment culture with a secondary enrichment medium to form a secondary enrichment culture and incubating the secondary enrichment culture for a second period of time; and

performing a test to detect the target bacterium using a second portion of the second enrichment culture.

Embodiment AC is a method of detecting a target bacterium, the method comprising:

mixing a first volume of a test sample with a second volume of a primary enrichment medium to form a primary enrichment culture;
wherein the primary enrichment culture comprises a redox dye;

incubating the primary enrichment culture for a first period of time equal to eight hours or less;

assessing the primary enrichment culture during the first period of time to detect whether the redox dye changes from a first state to a second state;

if the second state is not detected in the primary enrichment culture during the first period of time, incubating the primary enrichment culture for a third period of time; and

performing a test to detect the target bacterium using a third portion of the first enrichment culture.

Embodiment AD is the method of Embodiment AB or Embodiment AC, wherein the redox dye is selected from the group consisting of methylene blue, resazurin, and a tetrazolium dye.

Embodiment AE is the method of any one of Embodiments AB through AD, wherein a ratio of the first volume to the second volume is less than or equal to 10:1.

Embodiment AF is the method of any one of Embodiments AB through AE, wherein the primary enrichment medium is preheated.

Embodiment AG is the method of any one of Embodiments AB through AF, wherein incubating the primary enrichment culture for a first period of time comprises incubating the primary enrichment culture at 32-42° C., inclusive.

Embodiment AH is the method of any one of Embodiments AB through AG, wherein incubating the secondary enrichment culture for a second period of time comprises incubating the secondary enrichment culture at 32-42° C., inclusive.

Embodiment AI is the method of any one of Embodiments AB through AH, wherein incubating the primary enrichment culture for a third period of time comprises incubating the primary enrichment culture at 32-42° C., inclusive.

Embodiment AJ is the method of any one of Embodiments AB through AI, wherein the first state has a first color and the second state has a second color that is measurably different than first color.

Embodiment AK is the method of Embodiment AJ, wherein the first state is a colorless state and the second state is a colored state.

Embodiment AL is the method of Embodiment AJ, wherein the first state is a colored state and the second state is a colorless state.

Embodiment AM is the method of any one of Embodiments AB through AL, wherein the test sample comprises a component of the primary enrichment medium.

Embodiment AN is the method of any one of Embodiments AB through AM, wherein the primary enrichment medium is preheated to a temperature between 32° C. and 45° C., inclusive.

Embodiment AO is the method of any one of Embodiments AB through AN, wherein the first period of time is equal to or less than 4 hours.

Embodiment AP is the method of Embodiment AO, wherein the first period of time is equal to or less than 2 hours.

Embodiment AQ is the method of any one of Embodiments AB through AP, wherein assessing the primary enrichment culture during the first period of time comprises visually assessing the primary enrichment culture.

Embodiment AR is the method of any one of Embodiments AB through AQ, wherein assessing the primary enrichment culture during the first period of time comprises assessing the primary enrichment culture at a first time point and assessing the primary enrichment culture at a second time point after the first time point.

Embodiment AS is the method of Embodiment AR, wherein assessing the primary enrichment culture during the first period of time comprises assessing the primary enrichment culture for the first state of the redox dye at the first time point and assessing the primary enrichment culture for the second state of the redox dye at the second time point.

Embodiment AT is the method of one of Embodiments AB through AS, wherein assessing the primary enrichment culture during the first period of time comprises using an instrument to measure light absorbance or reflectance by the primary enrichment culture.

Embodiment AU is the method of any one of Embodiments AB through AT, wherein the first temperature, the second temperature, or the third temperature is a temperature between 32° C. and 43° C., inclusive.

Embodiment AV is the method of any one of Embodiments AB through AU, wherein the second period of time is about 10 hours to about 48 hours, inclusive.

Embodiment AW is the method of Embodiment AV, wherein the second period of time is about 10 hours to about 24 hours, inclusive.

Embodiment AX is the method of any one of Embodiments AC through AW, wherein the third period of time is about 10 hours to about 48 hours, inclusive.

Embodiment AY is the method of Embodiment AV, wherein the third period of time is about 10 hours to about 18 hours, inclusive.

Embodiment AZ is the method of any one of the preceding Embodiments, wherein performing a test to detect the target bacterium comprises performing a test selected from the group consisting of a test for detecting a predetermined enzyme activity associated with the target bacterium, an immunological test to detect an antigen associated with the target bacterium, a genetic test to detect a nucleic acid sequence associated with the target bacterium, culturing the bacterium on or in a semisolid medium, and a combination of any two or more of the foregoing tests.

Embodiment BA is the method of any one of Embodiments AB through AZ, wherein the primary enrichment medium is a liquid medium.

Embodiment BB is the method of Embodiment BA, wherein the secondary enrichment medium is a liquid medium.

Embodiment BC is a method of enriching a target microorganism in a liquid culture medium, the method comprising:

mixing a first volume of a test sample with a second volume of a primary enrichment medium to form a primary enrichment culture;
wherein the primary enrichment culture comprises a redox dye;

incubating the primary enrichment culture for a first period of time equal to eight hours or less;

assessing the primary enrichment culture during the first period of time to detect whether the redox dye changes from a first state to a second state;

if the second state is detected in the primary enrichment culture during the first period of time, mixing a first portion of the primary enrichment culture with a secondary enrichment medium to form a secondary enrichment culture and incubating the secondary enrichment culture for a second period of time.

Embodiment BD is the method of Embodiment BC, wherein the test sample is suspected of containing the target microorganism.

Objects and advantages of this invention are further illustrated by the following examples, but the particular materials and amounts thereof recited in these examples, as well as other conditions and details, should not be construed to unduly limit this invention.

### EXAMPLES

Materials

The primary enrichment medium, buffered peptone water (“BPW”; part number BP0115005), was obtained from 3M Health Care (St. Paul, Minn.). The secondary enrichment medium, Rappaport-Vassiliadis R10 broth (“RVR10”; part number BP0288500), was obtained from 3M Health Care. Raw chicken rinse samples were obtained from a commercial poultry processing plant. “Pre-intervention” rinses were obtained by washing defeathered chicken carcasses with BPW prior to subjecting them to a process intended to reduce the number of bacteria adhered to the carcasses. “Post-intervention” were obtained by washing defeathered chicken carcasses with BPW after subjecting them to a process intended to reduce the number of bacteria adhered to the carcasses. Plastic bags (Part No. BP134S, obtained from 3M Food Safety were used to hold the enrichment cultures during incubation. Methylene blue (part number M9140), was obtained from Sigma Chemical Company (St. Louis, Mo.). Molecular Detection System Salmonella detection kits (part no. MDAS96NA) and 3M PETRIFILM™ Aerobic Count Plates (“AC plates”) were obtained from 3M Company (St. Paul, Minn.).

### Example 1. Detection of Salmonella in Chicken Rinse Samples

Twenty randomized pre-intervention and post-intervention chicken rinse samples (30 mL per sample) each were placed into individual bags holding 30 mL of pre-warmed (41.5° C.) primary enrichment medium (BPW containing 20 mg/mL methylene blue) to form primary enrichment cultures. The liquid medium in each primary enrichment culture had a blue color. A small sample of each primary enrichment culture was serially-diluted and 1 mL of selected dilutions were used to determine the initial count of total aerobic microorganisms in PETRIFILM AC plates.

The primary enrichment cultures were incubated in an environmental chamber at 41.5° C. Each bag was visually inspected after 1, 2, 3, 4, 6, 10, and 18 hours of incubation to determine whether the methylene blue dye was converted from a blue (oxidized) form to a colorless (reduced) form. When conversion of the dye to a colorless state was observed in a bag within 4 hours, 100 microliters of the primary enrichment culture was removed 4.5 hours after formation of the primary enrichment culture and was mixed with 10 mL of sterile secondary enrichment medium to form a secondary enrichment culture. The secondary enrichment cultures were incubated at 41.5° C. for 18 hours. The remainder of the primary enrichment cultures (in which the redox dye had converted to colorless) were incubated at 41.5° C. for an additional 18 hours.

When conversion of the redox dye was not observed within 4 hours, the primary culture was allowed to continue incubating at 41.5° C. for an additional 18 hours. At the end of the 18-hour incubation periods, a 20 microliter sample from each primary or secondary enrichment culture was removed and processed to detect Salmonella microorganisms using the 3M Molecular Detection System according to the manufacturer's instructions. The results are shown in Table 1.

TABLE 1
Colony counts from PETRIFILM AC plates are shown
in Log CFU/mL. Detection of Salmonella was performed
with the 3M Molecular Detection System.

| | | Time to | | | |
|---|---|---|---|---|---|
| | | Redox | | | |
| | | Conversion | Trans- | Salmonella | Salmonella |
| | | in Primary | ferred to | Detection | Detection |
| Sam- | Log | Culture | Secondary | in Secondary | in Primary |
| ple | CFU/mL | (hr) | Culture | Culture | Culture |
| 1 | 7.25 | 2 | Yes | Positive | Negative |
| 2 | 6.31 | 4 | Yes | Negative | Negative |
| 3 | 4.70 | 18 | No | NT | Negative |
| 4 | 7.57 | 2 | Yes | Positive | Positive |
| 5 | 6.10 | 4 | Yes | Negative | Negative |
| 6 | 5.38 | 18 | No | NT | Negative |
| 7 | 8.40 | 1 | Yes | Positive | Negative |
| 8 | 7.08 | 3 | Yes | Positive | Positive |
| 9 | 6.18 | 4 | Yes | Positive | Negative |
| 10 | 5.51 | 6 | No | NT | Negative |
| 11 | 5.48 | 6 | No | NT | Negative |
| 12 | 5.34 | 6 | No | NT | Negative |
| 13 | NT | 18 | No | NT | Negative |
| 14 | NT | 18 | No | NT | Negative |
| 15 | 4.78 | 10 | No | NT | Negative |
| 16 | NT | 18 | No | NT | Negative |
| 17 | NT | 18 | No | NT | Negative |
| 18 | NT | 18 | No | NT | Negative |
| 19 | 4.30 | 10 | No | NT | Negative |
| 20 | 6.11 | 4 | Yes | Positive | Positive |

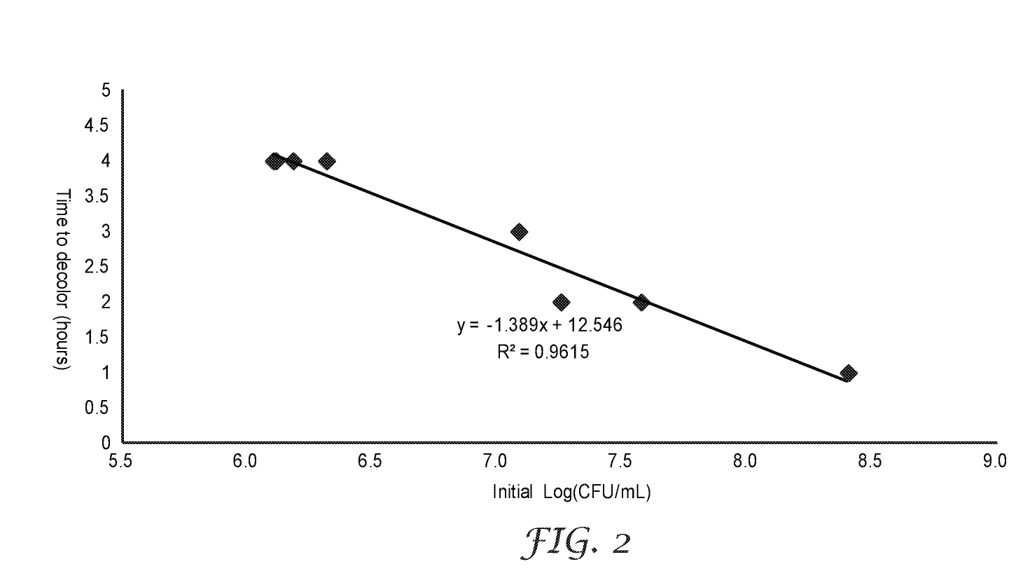

The correlation between the total aerobic count at inoculation of the primary enrichment cultures and the amount of time it took to convert the redox dye is shown in FIG. 2. The results indicate an inverse correlation between the initial bacterial count and the time required to convert the redox dye.

The data in Table 1 show that using the redox dye to determine when to transfer a portion of the primary enrichment culture to a secondary enrichment medium resulted in detection of Salmonella in three more samples (i.e., samples 1, 7, and 9) than would have otherwise been detected by testing the primary enrichment cultures.

The complete disclosure of all patents, patent applications, and publications, and electronically available material cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

Various modifications may be made without departing from the spirit and scope of the invention. These and other embodiments are within the scope of the following claims.

1. A method of detecting a bacterium belonging to the genus Salmonella, the method comprising:
mixing a first volume of a test sample with a second volume of a primary enrichment medium to form a primary enrichment culture;
wherein the primary enrichment culture comprises a redox dye;
incubating the primary enrichment culture for a first period of time equal to eight hours or less;
assessing the primary enrichment culture during the first period of time to detect whether the redox dye changes from a first state to a second state;
if the second state is detected in the primary enrichment culture during the first period of time, mixing a first portion of the primary enrichment culture with a secondary enrichment medium to form a secondary enrichment culture and incubating the secondary enrichment culture for a second period of time; and
performing a test to detect the bacterium of the genus Salmonella using a second portion of the second enrichment culture.

2. A method of detecting a bacterium belonging to the genus Salmonella, the method comprising:
mixing a first volume of a test sample with a second volume of a primary enrichment medium to form a primary enrichment culture;
wherein the primary enrichment culture comprises a redox dye;
incubating the primary enrichment culture for a first period of time equal to eight hours or less;
assessing the primary enrichment culture during the first period of time to detect whether the redox dye changes from a first state to a second state;
if the second state is not detected in the primary enrichment culture during the first period of time, incubating the primary enrichment culture for a third period of time; and
performing a test to detect the bacterium of the genus Salmonella using a third portion of the primary enrichment culture

3. The method of claim 1, wherein the redox dye is selected from the group consisting of methylene blue, resazurin, and tetrazolium

4. The method of claim 1, wherein a ratio of the first volume to the second volume is less than or equal to 10:1.

5. The method of claim 1, wherein the first state has a first color and the second state has a second color that is measurably different than first color.

6. The method of claim 5, wherein the first state is a colorless state and the second state is a colored state or wherein the first state is a colored state and the second state is a colorless state.

7. The method of claim 1, wherein the test sample comprises a component of the primary enrichment medium.

8. The method of claim 1, wherein the first period of time is equal to or less than 4 hours.

9. The method of claim 1, wherein assessing the primary enrichment culture during the first period of time comprises assessing the primary enrichment culture at a first time point and assessing the primary enrichment culture at a second time point after the first time point.

10. The method of claim 1, wherein assessing the primary enrichment culture during the first period of time comprises using an instrument to measure light absorbance or reflectance by the primary enrichment culture.

11. A method of detecting a target bacterium, the method comprising:
mixing a first volume of a test sample with a second volume of a primary enrichment medium to form a primary enrichment culture;
wherein the primary enrichment culture comprises a redox dye;
incubating the primary enrichment culture for a first period of time equal to eight hours or less;
assessing the primary enrichment culture during the first period of time to detect whether the redox dye changes from a first state to a second state;
if the second state is detected in the primary enrichment culture during the first period of time, mixing a first portion of the primary enrichment culture with a secondary enrichment medium to form a secondary enrichment culture and incubating the secondary enrichment culture for a second period of time; and
performing a test to detect the target bacterium using a second portion of the second enrichment culture.

12. The method of claim 11 wherein,
mixing a first volume of a test sample with a second volume of a primary enrichment medium to form a primary enrichment culture;
wherein the primary enrichment culture comprises a redox dye;
incubating the primary enrichment culture for a first period of time equal to eight hours or less;
assessing the primary enrichment culture during the first period of time to detect whether the redox dye changes from a first state to a second state;
if the second state is not detected in the primary enrichment culture during the first period of time, incubating the primary enrichment culture for a third period of time; and
performing a test to detect the target bacterium using a third portion of the first enrichment culture.

13. The method of claim 11, wherein the redox dye is selected from the group consisting of methylene blue, resazurin, and a tetrazolium dye.

14. The method of claim 11, wherein a ratio of the first volume to the second volume is less than or equal to 10:1.

15. The method of claim 11, wherein the first state has a first color and the second state has a second color that is measurably different than first color.

16. The method of claim 15, wherein the first state is a colorless state and the second state is a colored state or wherein the first state is a colored state and the second state is a colorless state.

17. The method of claim 11,
wherein the test sample comprises a component of the primary enrichment medium.

18. The method of claim 11, wherein the first period of time is equal to or less than 4 hours.

19. The method of claim 11, wherein assessing the primary enrichment culture during the first period of time comprises assessing the primary enrichment culture at a first time point and assessing the primary enrichment culture at a second time point after the first time point.

20. The method of claim 11, wherein assessing the primary enrichment culture during the first period of time comprises using an instrument to measure light absorbance or reflectance by the primary enrichment culture.
